(19) United States
(12) Patent Application Publication
(10) Pub. No.: US 2025/0362299 A1
(43) Pub. Date: Nov. 27, 2025

(54) DUAL-MODE IMMUNOASSAY STRUCTURE BASED ON TITANIUM CARBIDE SERS SUBSTRATE AND RARE-EARTH DOPED SODIUM YTTRIUM FLUORIDE NANOPARTICLES, AND ITS PREPARATION METHOD AND APPLICATION

(71) Applicant: The First Affiliated Hospital Of Ningbo University, Ningbo (CN)

(72) Inventors: Kerong Wu, Ningbo (CN); Tao Jiang, Ningbo (CN); Shiyuan Wang, Ningbo (CN)

(21) Appl. No.: 19/291,560

(22) Filed: Aug. 5, 2025

Related U.S. Application Data
(63) Continuation of application No. PCT/CN2024/095052, filed on May 24, 2024.

(30) Foreign Application Priority Data
May 24, 2024 (WO) PCT/CN2024/095052

(51) Int. Cl. G01N 33/574; G01N 33/543

(52) U.S. Cl. CPC G01N 33/57434; G01N 33/54346

(57) ABSTRACT

A dual-mode immunoassay structure includes a titanium carbide/molybdenum disulfide composite immunoassay substrate and a NaYF₄:Yb,Er immunoprobe, wherein the NaYF₄:Yb, Er comprises 78% Y, 20% Yb, and 2% Er. The method of preparation includes the steps of: (S10) preparing titanium carbide powder via an acid-etching method; (S20) fabricating the titanium carbide/molybdenum disulfide composite immunoassay substrate; and (S30) preparing the NaYF₄:Yb,Er immunoprobe. The dual-mode immunoassay structure is suitable for use in the detection of prostate cancer, providing both Surface-Enhanced Raman Scattering (SERS) and upconversion luminescence signals for analysis, rendering it applicable for clinical diagnostics.

### CROSS REFERENCE OF RELATED APPLICATION

This is a Continuation Application of PCT/CN2024/095052, filed May 24, 2024, wherein the entire content of which is expressly incorporated herein by reference.

### BACKGROUND OF THE PRESENT INVENTION

### Field of Invention

The present invention relates to the technical field of prostate cancer detection, and more particularly, to a dual-mode immunoassay structure based on a titanium carbide SERS substrate and rare-earth doped sodium yttrium fluoride nanoparticles, as well as a method for its preparation and application thereof.

### Description of Related Arts

In recent years, cancer has become a significant cause of premature death, posing a severe threat to human health. Prostate cancer is one of the most common malignancies and is recognized as the third leading cause of cancer-related mortality in men. Achieving early detection and dynamic monitoring is critical for the effective treatment of most cancer patients, wherein prostate-specific antigen (PSA) has been widely recognized as a key biomarker. However, current mainstream detection methods for PSA are relatively singular and suffer from issues such as long turnaround times, cumbersome operating procedures, and low detection accuracy. Compared to traditional detection methods, Surface-Enhanced Raman Scattering (SERS), as an efficient spectroscopic detection technique, offers extremely high detection sensitivity and molecular fingerprinting capabilities, showing great promise for applications in clinical diagnostics. Upconversion luminescent materials, which are anti-Stokes phosphors, are materials that emit visible light upon excitation with near-infrared light. Currently, upconversion luminescent materials are widely used in biofluorescence imaging, three-dimensional displays, and anti-counterfeiting applications. Upconversion luminescent materials typically consist of two parts: a crystal host material and doped rare-earth ions. Among these, fluorides and halides (oxyhalides) have low phonon energies and exhibit high upconversion luminous efficiency, making them the most common host materials for upconversion luminescence. At present, the preparation of rare-earth doped fluoride upconversion nanoluminescent materials yields materials with narrow absorption and emission spectra, making them particularly suitable for the detection of specific markers in the medical field.

However, there have been no reports to date of combining a titanium carbide-based SERS substrate with rare-earth doped sodium yttrium fluoride nanoparticles to achieve dual-mode detection of cancer biomarkers. In fact, a dual-mode detection technology based on upconversion luminescence and Surface-Enhanced Raman Scattering can allow the advantages of both detection methods to complement each other, further enhancing the accuracy and sensitivity of cancer detection.

### SUMMARY OF THE PRESENT INVENTION

An advantage of the present invention is to provide a dual-mode immunoassay structure based on a titanium carbide SERS substrate and rare-earth doped sodium yttrium fluoride nanoparticles, as well as its preparation method and application, which can improve the efficiency and sensitivity of cancer detection and is of great significance for the early screening of cancer.

Another advantage of the present invention is to provide a dual-mode immunoassay structure based on a titanium carbide SERS substrate and rare-earth doped sodium yttrium fluoride nanoparticles, and its preparation method and application, which combines the advantages of the dual spectra emitted by the dual-mode structure of the present invention, including the high efficiency, good stability, and strong anti-interference capability of upconversion luminescence, along with the strong fingerprinting capability and high detection sensitivity of SERS spectra, thereby significantly improving the accuracy of clinical detection for cancer biomarkers.

Another advantage of the present invention is to provide a dual-mode immunoassay structure based on a titanium carbide SERS substrate and rare-earth doped sodium yttrium fluoride nanoparticles, and its preparation method and application, wherein compared to traditional fluorescent materials, the upconversion material, as a fluorescent material that can emit short-wavelength light under red or infrared light excitation, converts low-energy, long-wavelength light into high-energy, short-wavelength light. It also possesses narrow absorption and emission spectra, making it particularly suitable for the detection of specific markers in the medical field.

Another advantage of the present invention is to provide a dual-mode immunoassay structure based on a titanium carbide SERS substrate and rare-earth doped sodium yttrium fluoride nanoparticles, and its preparation method and application, wherein SERS technology, as another efficient spectroscopic detection technique, has extremely high detection sensitivity and molecular fingerprinting capabilities, also demonstrating promising application prospects in clinical detection and being of great significance for improving the accuracy and sensitivity of cancer detection.

Another advantage of the present invention is to provide a dual-mode immunoassay structure based on a titanium carbide SERS substrate and rare-earth doped sodium yttrium fluoride nanoparticles, and its preparation method and application, wherein a Raman spectrometer is used to perform fluorescence spectroscopy or Raman spectroscopy measurements on the sandwich immunoassay structure obtained after the aforementioned immunoassay reaction, and the concentration of the cancer biomarker antigen to be measured is calculated based on the linear relationship between the antigen concentration and the intensity of the characteristic fluorescence or Raman peaks, yielding relatively accurate results.

Another advantage of the present invention is to provide a dual-mode immunoassay structure based on a titanium carbide SERS substrate and rare-earth doped sodium yttrium fluoride nanoparticles, and its preparation method and application, wherein the preparation method is simple, low-cost, convenient to use, and suitable for widespread clinical application.

Another advantage of the present invention is to provide a dual-mode immunoassay structure based on a titanium carbide SERS substrate and rare-earth doped sodium yttrium fluoride nanoparticles, and its preparation method and application, wherein Surface-Enhanced Raman Scattering, in combination with the dual-mode structure of the present invention, allows for an increase in the accuracy of the dual-mode detection technology.

According to one aspect of the present invention, a method for preparing a dual-mode immunoassay structure based on a titanium carbide SERS substrate and rare-earth doped sodium yttrium fluoride nanoparticles is provided, comprising the following steps:
(S10) preparing titanium carbide powder using an acid-etching method;
(S20) preparing a titanium carbide/molybdenum disulfide composite immunoassay substrate; and
(S30) preparing a NaYF₄:Yb,Er immunoprobe.

Wherein the step (S10) comprises the following sub-steps: (S101) preparing a hydrochloric acid solution containing lithium fluoride; (S102) adding titanium aluminum carbide powder to the aforementioned solution and reacting to obtain a titanium carbide solution; (S103) centrifugally washing the titanium carbide solution with dilute hydrochloric acid, followed by washing with deionized water, subjecting the washed solution to ultrasonication in an ice bath, and centrifuging to collect the supernatant; and

(S104) drying the supernatant to obtain titanium carbide (MXene) powder.

Wherein in the step (S101), 1-3 g of lithium fluoride is added to 20 mL of 9 M hydrochloric acid and stirred continuously for 10 minutes at room temperature; in the step (S102), 1-3 g of titanium aluminum carbide powder is slowly added to the solution containing lithium fluoride and hydrochloric acid with stirring, heated in a water bath to 45° C., and reacted continuously for 24-48 hours to obtain the titanium carbide solution; in the step (S103), the titanium carbide solution is centrifugally washed three times with 1 M dilute hydrochloric acid, and then repeatedly washed with deionized water until the pH of the supernatant is 6-7.

Wherein the step (S20) comprises the following sub-steps: (S201) dissolving ammonium molybdate and thiourea in deionized water, then adding titanium carbide, and ultrasonically dispersing to form a mixed liquid; (S202) placing the mixed solution into a Teflon-lined stainless-steel autoclave, heating, and after the reaction is complete, cooling and centrifuging to obtain a black precipitate; (S203) washing the black precipitate with ethanol and deionized water, and vacuum drying to obtain a titanium carbide/molybdenum disulfide (MXene/MoS₂) composite; (S204) adding the MXene/MoS2 composite from step (S203) to deionized water to form a solution, drop-casting the solution onto a silicon wafer, soaking the silicon wafer in DMF, and then washing with a phosphate buffer solution; (S205) drop-casting a phosphate buffer solution of NHS/EDC onto the titanium carbide/molybdenum disulfide composite substrate; and (S206) drop-casting a solution containing an antibody, incubating, and washing to remove excess unreacted antibody to obtain the MXene/MoS₂ composite immunoassay substrate.

Wherein in the step (S201), the amount of ammonium molybdate is 15-20 mg, the amount of thiourea is 30-50 mg, and the amount of titanium carbide is 15-25 mg; in the step (S202), the mixed solution is placed in a Teflon-lined stainless-steel autoclave, heated to 200° C. for 24 hours, and after the reaction is complete, naturally cooled to room temperature; in the step (S203), the resulting a black precipitate is washed 4-6 times with ethanol and deionized water, then vacuum dried at 60° C. for 12 hours to obtain the titanium carbide/molybdenum disulfide (MXene/MoS₂) composite.

Wherein in a step (S204), the MXene/MoS₂ composite is added to deionized water at a mass-to-volume ratio of 1 mg: 100 μL to form a solution, 10-30 μL of the solution is drop-cast onto a silicon wafer, the silicon wafer is soaked in DMF for 2 hours, and then washed multiple times with a phosphate buffer solution; in the step (S205), 1 mL of a phosphate buffer solution of NHS/EDC is drop-cast onto the titanium carbide/molybdenum disulfide composite substrate, wherein the ratio of NHS to EDC is 1:1 and the concentration is 10 mg/mL; in the step (S206), 10 μL of a solution containing a PSA antibody is drop-cast, incubated at room temperature for 2 hours, washed to remove excess unreacted antibody, and stored at 4° C. to obtain the MXene/MoS₂ composite immunoassay substrate.

Wherein the step (S30) comprises the following sub-steps: (S301) mixing and stirring rare earth nitrates RE(NO₃)₃·6H₂O with an aqueous solution of citric acid; (S302) adding a sodium hydroxide solution with stirring, and then adding a sodium fluoride solution to obtain a colloidal suspension; (S303) transferring the colloidal suspension to a Teflon-lined stainless-steel reactor for reaction, and after the reaction is complete, cooling and centrifuging to obtain the reactant product; (S304) washing the product with ethanol and deionized water, and drying; (S305) mixing and reacting a solution of NaYF₄:Yb,Er in an R6G solution; (S306) washing away the excess R6G, then adding a phosphate buffer solution of NHS/EDC and incubating; (S307) after rinsing with a phosphate buffer, adding a solution containing an antibody, incubating, and washing to remove excess unreacted antibody to obtain the NaYF₄:Yb,Er (78% Y, 20% Yb, 2% Er) immunoprobe.

Wherein in the step (S301), the rare earth nitrate is RE(NO₃)₃·6H₂O (RE=78% Y, 20% Yb, 2% Er); in the step (S302), the resulting colloidal suspension is stirred for an additional 30 minutes before being transferred to a Teflon-lined stainless-steel reactor, reacted at 180° C. for 12 hours, and after the reaction is complete, naturally cooled to room temperature; in the step (S304), the reactant product is obtained by centrifugation, washed repeatedly 6 times with ethanol and deionized water, and the product is dried at 80° C. for 12 hours; in the step (S305), the NaYF₄:Yb,Er solution is mixed and reacted in 5-10 mL of a 1 mM R6G solution for 15 minutes, and then the excess R6G is washed away; in the step (S306), the ratio of NHS to EDC is 1:1, and the concentration is 10 mg/mL.

Wherein in the step (S307), a solution containing a PSA antibody is added, incubated for 1 hour, and washed to remove excess unreacted antibody to obtain the NaYF₄:Yb,Er immunoprobe.

The method further comprises a step (S40) of assembling a cancer biomarker detection system: a phosphate buffer solution containing the antigen to be measured is drop-cast onto the MXene/MoS₂ composite immunoassay substrate and allowed to stand, permitting an immunoassay reaction between the antigen and the antibody, followed by washing to remove the excess unreacted antigen; then, the NaYF₄:Yb, Er immunoprobe is drop-cast onto the MXene/MoS₂ composite immunoassay substrate with the adsorbed antigen, reacted at 37° C., and washed to remove the excess unreacted NaYF₄:Yb,Er immunoprobe, thereby obtaining the cancer biomarker detection system based on MXene/MoS₂ and NaYF₄:Yb,Er.

According to another aspect of the present invention, a dual-mode immunoassay structure based on a titanium carbide SERS substrate and rare-earth doped sodium yttrium fluoride nanoparticles is provided, characterized in that it comprises: a titanium carbide/molybdenum disulfide composite immunoassay substrate and a NaYF₄:Yb,Er immunoprobe, wherein the NaYF₄:Yb, Er comprises 78% Y, 20% Yb, and 2% Er.

According to yet another aspect of the present invention, an application of a dual-mode immunoassay structure based on a titanium carbide SERS substrate and rare-earth doped sodium yttrium fluoride nanoparticles is provided, wherein the dual-mode immunoassay structure based on a titanium carbide SERS substrate and rare-earth doped sodium yttrium fluoride nanoparticles is suitable for use in the detection of prostate cancer, colorectal cancer, ovarian cancer, or pancreatic cancer.

Wherein the dual-mode immunoassay structure based on a titanium carbide SERS substrate and rare-earth doped sodium yttrium fluoride nanoparticles comprises a titanium carbide/molybdenum disulfide composite immunoassay substrate and a NaYF₄:Yb,Er immunoprobe, wherein the NaYF₄:Yb,Er comprises 78% Y, 20% Yb, and 2% Er, and its preparation method comprises the steps of: (S10) preparing titanium carbide powder using an acid-etching method; (S20) preparing a titanium carbide/molybdenum disulfide composite immunoassay substrate; and (S30) preparing a NaYF₄:Yb,Er immunoprobe.

Wherein, in the application of the dual-mode immunoassay structure based on a titanium carbide SERS substrate and rare-earth doped sodium yttrium fluoride nanoparticles, a phosphate buffer solution containing the antigen to be measured is drop-cast onto the MXene/MoS₂ composite immunoassay substrate, and it is allowed to stand at 37° C. for 2 hours to allow for an immunoassay reaction between the antigen and antibody, followed by washing to remove the excess unreacted antigen; then, 20-40 μL of the NaYF₄:Yb,Er immunoprobe is drop-cast onto the MXene/MoS₂ composite immunoassay substrate with the adsorbed antigen, reacted at 37° C. for 2 hours, and washed to remove the excess unreacted NaYF₄:Yb,Er immunoprobe, thereby obtaining the cancer biomarker detection system based on MXene/MoS₂ and NaYF₄:Yb,Er; a Raman spectrometer is used to perform spectroscopic measurements on the composite of the NaYF₄:Yb,Er immunoprobe and the MXene/MoS₂ immunoassay substrate obtained after the immunoassay reaction, and the concentration of the cancer biomarker antigen to be measured is calculated based on the linear relationship between the antigen concentration and the intensity of a characteristic Raman peak.

### BRIEF DESCRIPTION OF THE DRAWINGS

FIG. 1 is a scanning electron microscope (SEM) image of the MXene/MoS₂ composite immunoassay substrate prepared in Example 1.

FIG. 2 is an SEM image of the MXene/MoS₂ composite immunoassay substrate prepared in Example 2.

FIG. 3 is an SEM image of the MXene/MoS₂ composite immunoassay substrate prepared in Example 3.

FIG. 4 is an SEM image of the NaYF₄:Yb, Er immunoprobe prepared in Example 1.

FIG. 5 is an SEM image of the NaYF₄:Yb, Er immunoprobe prepared in Example 2.

FIG. 6 is an SEM image of the NaYF₄:Yb, Er immunoprobe prepared in Example 3.

FIG. 7 is a SERS spectrum from the detection of different concentrations of prostate cancer using the MXene/MoS₂—NaYF₄:Yb,Er nanomaterials prepared in Example 1.

FIG. 8 is a SERS spectrum from the detection of different concentrations of prostate cancer using the MXene/MoS₂—NaYF₄:Yb,Er nanomaterials prepared in Example 2.

FIG. 9 is a SERS spectrum from the detection of different concentrations of prostate cancer using the MXene/MoS₂—NaYF₄:Yb,Er nanomaterials prepared in Example 3.

FIG. 10 is an upconversion spectrum from the detection of different concentrations of prostate cancer using the MXene/MoS₂—NaYF₄:Yb,Er nanomaterials prepared in Example 1.

FIG. 11 is an upconversion spectrum from the detection of different concentrations of prostate cancer using the MXene/MoS₂—NaYF₄:Yb,Er nanomaterials prepared in Example 2.

FIG. 12 is an upconversion spectrum from the detection of different concentrations of prostate cancer using the MXene/MoS₂—NaYF₄:Yb,Er nanomaterials prepared in Example 3.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

### Example 1

A method for preparing a dual-mode immunoassay structure based on a titanium carbide SERS substrate and rare-earth doped sodium yttrium fluoride nanoparticles, comprising the following steps:

### (1) Preparation of Titanium Carbide Powder by an Acid-Etching Method.

Add 1 g of lithium fluoride to 20 mL of hydrochloric acid (9 mmol/mL) and stir continuously for 10 minutes at room temperature. Slowly add 1 g of titanium aluminum carbide powder to the solution containing lithium fluoride and hydrochloric acid with stirring. Heat the mixture in a water bath to 45° C. and continue the reaction for 24 hours to obtain a titanium carbide solution. Centrifugally wash the titanium carbide solution three times with dilute hydrochloric acid (1 mmol/mL), and then repeatedly wash with deionized water until the pH of the supernatant is 6-7. Subject the washed solution to ultrasonication in an ice bath for 1 hour. Centrifuge the sonicated solution at 6000 rpm for 10 minutes to collect the supernatant. Then, dry the supernatant at 60° C. for 12 hours to obtain titanium carbide (MXene) powder.

### (2) Preparation of the Titanium Carbide/Molybdenum Disulfide Composite Immunoassay Substrate.

A. Dissolve 15 mg of ammonium molybdate and 30 mg of thiourea in 4 mL of deionized water. Slowly add this solution to 4 mL of a solution containing 15 mg of dissolved titanium carbide. Ultrasonically disperse for 10 minutes. Place the mixed solution in a Teflon-lined stainless-steel autoclave, heat to 200° C. for 24 hours. After the reaction is complete, naturally cool to room temperature and centrifuge. Wash the resulting black precipitate 4-6 times with ethanol and deionized water. Dry under vacuum at 60° C. for 12 hours to obtain the titanium carbide/molybdenum disulfide (MXene/MoS₂) composite.

B. Add the MXene/MoS₂ composite from step (2)A to deionized water at a mass-to-volume ratio of 1 mg: 100 μL to form a solution. Drop-cast 10 μL of solution onto a silicon wafer. Soak the silicon wafer in DMF for 2 hours, then wash multiple times with phosphate buffer solution. Subsequently, drop-cast 1 mL of a phosphate buffer of NHS/EDC (1:1, 10 mg/mL) onto the titanium carbide/molybdenum disulfide composite substrate. Then, drop-cast 10 μL of a solution containing PSA antibody, incubate at room temperature for 2 hours, wash to remove excess unreacted antibody, and store at 4° C. to obtain the MXene/MoS₂ composite immunoassay substrate.

### (3) Preparation of NaYF₄:Yb,Er (78% Y, 20% Yb, 2% Er) Immunoprobe.

A. First, mix 1 mmol of rare earth nitrate RE(NO₃)₃·6H₂O (RE=78% Y, 20% Yb, 2% Er) with 10 mL of aqueous citric acid solution (0.4 mmol/mL) and stir for 1 hour. Then, add 0.2 mL of sodium hydroxide solution (5 mmol/mL) and stir for 15 minutes. Add 8 mL of sodium fluoride solution (1 mmol/mL) to obtain a colloidal suspension. Continue stirring the resulting a colloidal suspension for 30 minutes, then transfer it to a 40 mL Teflon-lined stainless-steel reactor. React at 180° C. for 12 hours. After the reaction is complete, cool naturally to room temperature. Obtain the reactant product by centrifugation, wash it repeatedly 6 times with ethanol and deionized water, and dry the product at 80° C. for 12 hours.

B. Mix the NaYF₄:Yb, Er solution in 5 mL of R6G solution (1 mmol/mL) and react for 15 minutes. Wash away the excess R6G. Subsequently, add 1 mL of a phosphate buffer solution of NHS/EDC (1:1, 10 mg/mL), incubate at 37° C. for 1 hour. Rinse with phosphate buffer, then add a solution containing PSA antibody, incubate for 1 hour, and wash to remove excess unreacted antibody to obtain the NaYF₄:Yb, Er immunoprobe.

### (4) Assembly of the Cancer Biomarker Detection System.

Drop-cast a phosphate buffer solution containing the antigen to be measured onto the MXene/MoS₂ composite immunoassay substrate. Place it at 37° C. and allow to stand for 2 hours to ensure a complete immunoassay reaction between the antigen and antibody. Wash to remove the excess unreacted antigen. Then, drop-cast 20 μL of the NaYF₄:Yb,Er immunoprobe on to the MXene/MoS₂ composite immunoassay substrate with the adsorbed antigen, react at 37° C. for 2 hours, and wash to remove the excess unreacted NaYF₄:Yb,Er immunoprobe, thereby obtaining the cancer biomarker detection system based on MXene/MoS₂ and NaYF₄:Yb,Er. Use a Raman spectrometer to perform spectroscopic measurements on the composite of the NaYF₄:Yb, Er immunoprobe and the MXene/MoS₂ immunoassay substrate obtained after the immunoassay reaction. Calculate the concentration of the antigen to be measured based on the linear relationship between the antigen concentration and the intensity of a characteristic Raman peak.

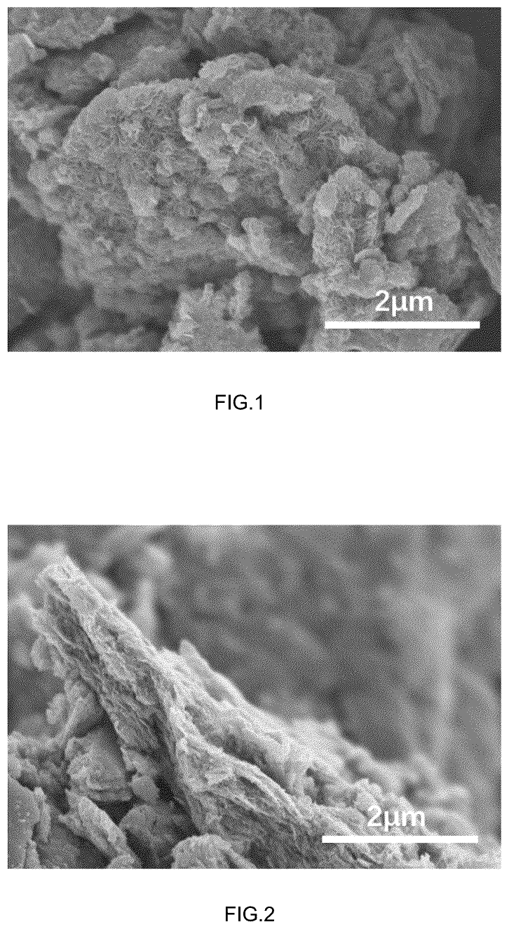

FIG. 1 shows a scanning electron microscope image of the MXene/MoS₂ composite prepared in this example. As can be seen from FIG. 1, web-like MoS₂ encapsulates MXene flakes, forming a composite nanostructure.

FIG. 4 shows the NaYF₄:Yb, Er upconversion luminescent nanoparticles prepared in this example. As can be seen from FIG. 4, the upconversion luminescent nanoparticles exhibit a quasi-spherical shape.

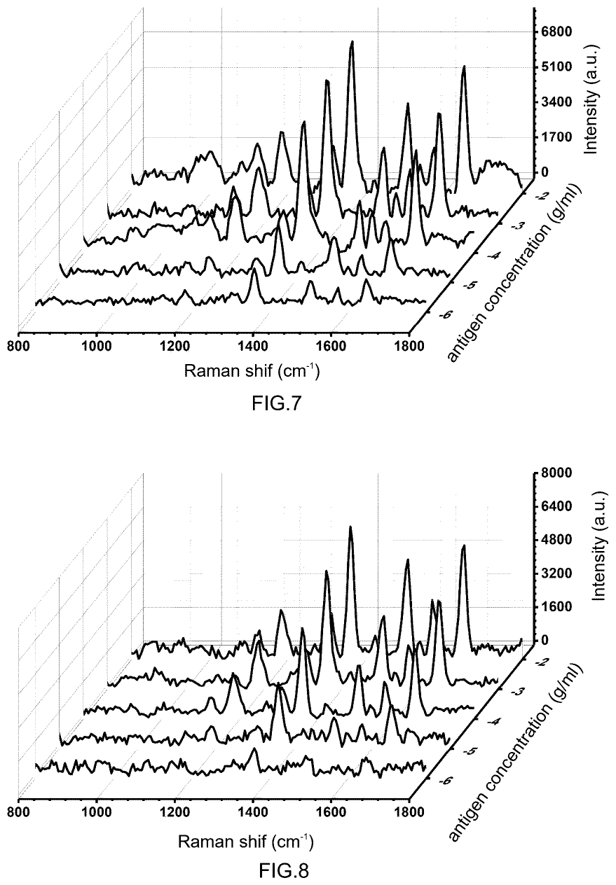

FIG. 7 shows the Raman spectra obtained by performing Raman detection on the substrate after the immunoassay reaction. The reaction involved the NaYF₄:Yb,Er upconversion luminescent nanoparticle immunoprobe and the MXene/MoS₂ composite SERS immunoassay substrate prepared in this example with different concentrations of the target antigen (concentrations from 10⁻² mg/mL to 10⁻⁶ mg/mL). As can be seen from the figure, as the concentration of the target antigen decreases, the intensity of the a characteristic Raman peaks of the label molecule gradually decreases. When the a target antigen concentration is reduced to 10⁻⁶ mg/mL, the characteristic Raman peak of the label molecule is still clearly distinguishable from the background Detections, this concentration is the detection limit for the antigen in this scheme.

FIG. 10 shows the upconversion spectra obtained by performing luminescence detection on the substrate after the immunoassay reaction. The reaction involved the NaYF₄:Yb,Er upconversion luminescent nanoparticle immunoprobe and the MXene/MoS₂ composite SERS immunoassay substrate prepared in this example with different concentrations of the target antigen (concentrations from 10⁻² mg/mL to 10⁻⁶ mg/mL). As can be seen from the figure, as the concentration of the target antigen decreases, the intensity of the upconversion emission spectrum gradually decreases. When the target antigen concentration is reduced to 10⁻⁶ mg/mL, the upconversion emission peak is still clearly distinguishable from the background signals, este and this concentration represents the detection limit for the antigen in this scheme.

### Example 2

A method for preparing a dual-mode immunoassay structure based on a titanium carbide SERS substrate and rare-earth doped sodium yttrium fluoride nanoparticles, comprising the following steps:

### (1) Preparation of Titanium Carbide Powder by an Acid-Etching Method.

Add 2 g of lithium fluoride to 20 mL of hydrochloric acid (9 mmol/mL) and stir continuously for 10 minutes at room temperature. Slowly add 2 g of titanium aluminum carbide powder to the solution containing lithium fluoride and hydrochloric acid with stirring. Heat the mixture in a water bath to 45° C. and continue the reaction for 36 hours to obtain a titanium carbide solution. Centrifugally wash the titanium carbide solution three times with dilute hydrochloric acid (1 mmol/mL), and then repeatedly wash with deionized water until the pH of the supernatant is 6-7. Subject the washed solution to ultrasonication in an ice bath for 1 hour. Centrifuge the sonicated solution at 6000 rpm for 10 minutes to collect the supernatant. Then, dry the supernatant at 60° C. for 12 hours to obtain titanium carbide (MXene) powder.

### (2) Preparation of the Titanium Carbide/Molybdenum Disulfide Composite Immunoassay Substrate.

A. Dissolve 17 mg of ammonium molybdate and 40 mg of thiourea in 4 mL of deionized water. Slowly add this solution to 4 mL of a solution containing 20 mg of dissolved titanium carbide. Ultrasonically disperse for 10 minutes. Place the mixed solution in a Teflon-lined stainless-steel autoclave, heat to 200° C. for 24 hours. After the reaction is complete, naturally cool to room temperature and centrifuge. Wash the resulting black precipitate 4-6 times with ethanol and deionized water. Dry under vacuum at 60° C. for 12 hours to obtain the titanium carbide/molybdenum disulfide (MXene/MoS₂) composite.

B. Add the MXene/MoS₂ composite from step (2)A to deionized water at a mass-to-volume ratio of 1 mg: 100 μL to form a solution. Drop-cast 20 μL of solution onto a silicon wafer. Soak the silicon wafer in DMF for 2 hours, then wash multiple times with phosphate buffer solution. Subsequently, drop-cast 1 mL of a phosphate buffer of NHS/EDC (1:1, 10 mg/mL) onto the titanium carbide/molybdenum disulfide composite substrate. Then, drop-cast 10 μL of a solution a PSA antibody, incubate at room temperature for 2 hours, wash to remove excess unreacted antibody, and store at 4° C. to obtain the MXene/MoS₂ composite immunoassay substrate.

### (3) Preparation of NaYF₄:Yb,Er (78% Y, 20% Yb, 2% Er) Immunoprobe.

A. First, mix 2 mmol of rare earth nitrate RE(NO₃)₃·6H₂O (RE=78% Y, 20% Yb, 2% Er) a with 10 mL of aqueous citric acid solution (0.4 mmol/mL) and stir for 1 hour. Then, add 0.2-0.4 mL of sodium hydroxide solution (5 mmol/mL) and stir for 15 minutes. Add 9 mL of sodium fluoride solution (1 mmol/mL) to obtain a colloidal suspension. Continue stirring the resulting a colloidal suspension for 30 minutes, then transfer it to a 40 mL Teflon-lined stainless-steel reactor. React at 180° C. for 12 hours. After the reaction is complete, cool naturally to room temperature. Obtain the reactant product by centrifugation, wash it repeatedly 6 times with ethanol and deionized water, and dry the product at 80° C. for 12 hours.

B. Mix the NaYF₄:Yb,Er solution in a 7 mL R6G solution (1 mM) and react for 15 minutes. Wash away the excess R6G. Subsequently, add 1 mL of a phosphate buffer solution of NHS/EDC (1:1, 10 mg/mL), incubate at 37° C. for 1 hour. Rinse with phosphate buffer, then add a solution containing PSA antibody, incubate for 1 hour, and wash to remove excess unreacted antibody to obtain the NaYF₄:Yb,Er immunoprobe.

### (4) Assembly of the Cancer Biomarker Detection System.

A phosphate buffer solution containing the antigen to be measured is drop-cast onto the MXene/MoS₂ composite immunoassay substrate. This is allowed to stand at 37° C. for 2 hours to allow for a complete immunoassay reaction between the antigen and the antibody. The substrate is then washed to remove any excess unreacted antigen. Subsequently, 30 μL of the NaYF₄:Yb,Er immunoprobe is drop-cast onto the MXene/MoS₂ composite immunoassay substrate, which now has the antigen adsorbed to it. This is allowed to react at 37° C. for 2 hours and is then washed to remove the excess unreacted NaYF₄:Yb, Er immunoprobe, thereby obtaining the cancer biomarker detection system based on MXene/MoS₂ and NaYF₄:Yb,Er. A Raman spectrometer is used to perform spectroscopic measurements on the composite of the NaYF₄:Yb, Er immunoprobe and the MXene/MoS₂ immunoassay substrate obtained after the aforementioned immunoassay reaction. The concentration of the cancer biomarker antigen to be measured is calculated based on the linear relationship between the antigen concentration and the intensity of a characteristic Raman peak.

FIG. 2 shows a scanning electron microscope image of the MXene/MoS₂ composite prepared in this example. As can be seen from FIG. 2, web-like MoS₂ encapsulates MXene flakes, forming a composite nanostructure.

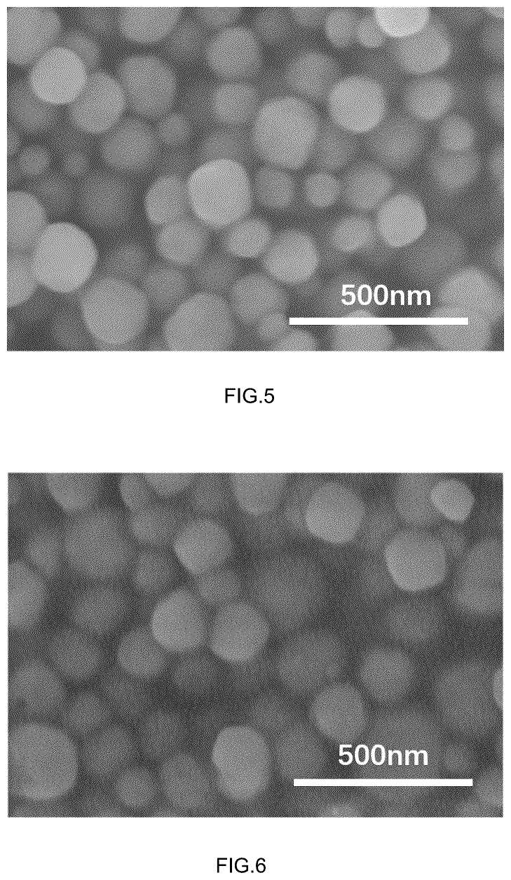

FIG. 5 shows the NaYF₄:Yb, Er upconversion luminescent nanoparticles a prepared in this example. As can be seen from FIG. 5, the upconversion luminescent nanoparticles exhibit a quasi-spherical shape.

FIG. 8 shows the results similar to those described for FIG. 7, but using the materials prepared according to this Example. As can be seen from the figure, as the concentration of the target antigen decreases, the intensity of the Raman spectrum of the label molecule gradually decreases. A distinct Raman peak of the label molecule relative to the background signal can still be seen when the antigen concentration is decreased to 10-6 mg/mL, which represents the detection limit of this configuration.

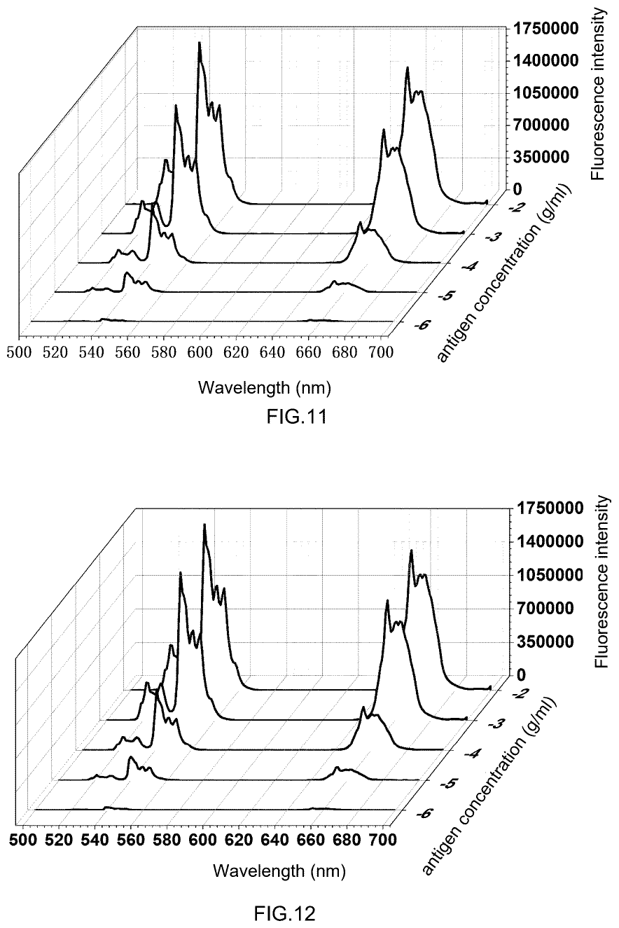

FIG. 11 shows the results similar to those described for FIG. 10, but a using the materials prepared according to this Example. As can be seen from the figure, as the concentration of the target antigen decreases, the intensity of the upconversion emission spectrum gradually decreases. A distinct upconversion emission peak relative to the background signal can still be observed when the target antigen concentration is decreased to 10-6 mg/mL, this being the detection limit of this configuration.

### Example 3

A method for preparing a dual-mode immunoassay structure based on a titanium carbide SERS substrate and rare-earth doped sodium yttrium fluoride nanoparticles, comprising the following steps:

### (1) Preparation of Titanium Carbide Powder by an Acid-Etching Method.

3 g of lithium fluoride was added to 20 mL of hydrochloric acid (9 mmol/mL) and stirred continuously for 10 minutes at room temperature. While stirring, 3 g of titanium aluminum carbide powder was slowly added to the solution containing lithium fluoride and hydrochloric acid. The mixture was heated in a water bath to 45° C. and the reaction was continued for 48 hours to obtain a titanium carbide solution. The titanium carbide solution was centrifugally washed three times with dilute hydrochloric acid (1 mmol/mL), and then washed repeatedly with deionized water until the supernatant reached a pH of 6-7. The washed solution was subjected to ultrasonication in an ice bath for 1 hour. The sonicated solution was then centrifuged at 6000 rpm for 10 minutes to obtain the supernatant. Thereafter, the supernatant was dried at 60° C. for 12 hours to obtain titanium carbide (MXene) powder.

### (2) Preparation of the Titanium Carbide/Molybdenum Disulfide Composite Immunoassay Substrate.

A. Take 20 mg of ammonium molybdate and 50 mg of thiourea and dissolve them in 4 mL of deionized water. This solution is then slowly added to 4 mL of a solution containing 25 mg of dissolved titanium carbide and ultrasonically dispersed for 10 minutes. The resulting mixed solution is placed in a Teflon-lined stainless-steel autoclave and heated to 200° C. for 24 hours. After the reaction is complete, it is allowed to cool naturally to room temperature and then centrifuged. The resulting black precipitate is washed 4-6 times with ethanol and deionized water, and subsequently dried under vacuum at 60° C. for 12 hours to obtain the titanium carbide/molybdenum disulfide (MXene/MoS₂) composite.

B. The MXene/MoS₂ composite from step (2)A is added to deionized water at a mass-to-volume ratio of 1 mg: 100 μL to form a solution. From 10-30 μL of this solution is drop-cast onto a silicon wafer. The silicon wafer is soaked in DMF for 2 hours and then washed multiple times with a phosphate buffer solution. Subsequently, 1 mL of an NHS/EDC phosphate buffer solution (1:1, 10 mg/mL) is drop-cast onto the titanium carbide/molybdenum disulfide composite substrate. Thereafter, 10 μL of a solution containing PSA antibody is drop-cast, incubated at room temperature for 2 hours, and then washed to remove excess unreacted antibody. The resulting MXene/MoS₂ composite immunoassay substrate is stored at 4° C.

### (3) Preparation of the NaYF₄:Yb,Er (78% Y, 20% Yb, 2% Er) Immunoprobe.

A. First, 3 mmol of rare earth nitrate RE(NO₃)₃·6H₂O (where RE=78% Y, 20% Yb, 2% Er) is mixed with 10 mL of an aqueous citric acid solution (0.4 mmol/mL) and stirred for 1 hour. Then, 0.4 mL of a sodium hydroxide solution (5 mmol/mL) is added, and the mixture is stirred for 15 minutes. This is followed by the addition of 10 mL of a sodium fluoride solution (1 mmol/mL) to obtain a colloidal suspension. The resulting colloidal suspension is stirred for an additional 30 minutes and then transferred to a 40 mL Teflon-lined stainless-steel reactor. The reaction is carried out at 180° C. for 12 hours. After completion, the reactor is allowed to cool naturally to room temperature. The reactant product is obtained by centrifugation, washed repeatedly 6 times with ethanol and deionized water, and the final product is dried at 80° C. for 12 hours.

B. The NaYF₄:Yb,Er solution is mixed in 5-10 mL of an R6G solution (1 mM) and allowed to react for 15 minutes. The excess R6G is washed away. Subsequently, 1 mL of an NHS/EDC phosphate buffer solution (1:1, 10 mg/mL) is added, and the mixture is incubated at 37° C. for 1 hour. After being rinsed with a phosphate buffer, a solution containing PSA antibody is added and incubated for 1 hour. The product is then washed to remove excess unreacted antibody to yield the final NaYF₄:Yb,Er immunoprobe.

### (4) Assembly of the Cancer Biomarker Detection System.

A phosphate buffer solution containing the antigen to be measured is drop-cast onto the MXene/MoS₂ composite immunoassay substrate. This is allowed to stand at 37° C. for 2 hours to facilitate a complete immunoassay reaction between the antigen and the antibody. The substrate is then washed to remove any excess unreacted antigen. Subsequently, 40 μL of the NaYF₄:Yb, Er immunoprobe is drop-cast onto the MXene/MoS₂ composite immunoassay substrate, which now has the antigen adsorbed to it. This is allowed to react at 37° C. for 2 hours. The substrate is then washed to remove excess unreacted NaYF₄:Yb,Er immunoprobe, thereby obtaining the a cancer biomarker detection system based on MXene/MoS₂ and NaYF₄:Yb,Er. A Raman spectrometer is used to perform spectroscopic measurements on the composite formed between the NaYF₄:Yb,Er immunoprobe and the MXene/MoS₂ immunoassay substrate after the immunoassay reaction. The concentration of the antigen being tested is calculated based on the linear relationship between the cancer biomarker antigen concentration and the intensity of the characteristic Raman peak.

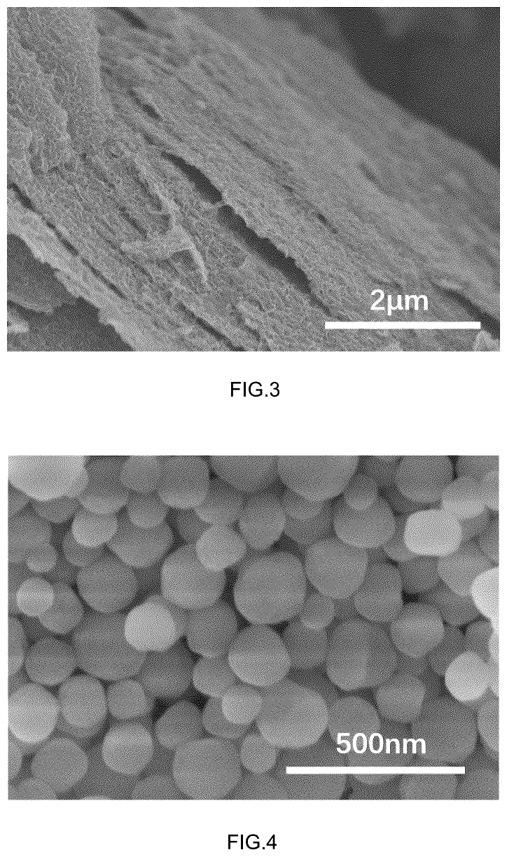

FIG. 3 shows a scanning electron microscope image of the MXene/MoS₂ composite prepared in this example. As can be seen from FIG. 3, web-like MoS₂ encapsulates MXene flakes, forming a composite nanostructure.

FIG. 6 shows the NaYF₄:Yb, Er upconversion luminescent nanoparticles prepared in this example. As can be seen from FIG. 6, the upconversion luminescent nanoparticles exhibit a quasi-spherical shape.

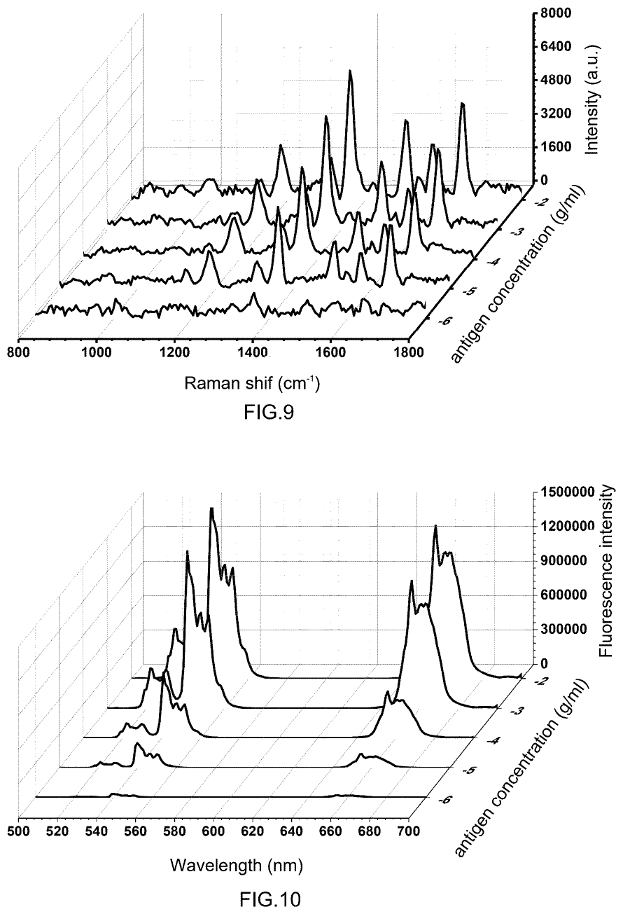

FIG. 9 is a Raman spectrum obtained by performing Raman detection on the substrate after an immunoassay reaction between the NaYF₄:Yb,Er upconversion luminescent nanoparticle immunoprobe and the MXene/MoS₂ composite SERS immunoassay substrate prepared in this example with different concentrations of the target antigen (ranging from 10-2 mg/mL to 10-6 mg/mL). As can be seen from the figure, as the concentration of the target antigen decreases, the intensity of the characteristic Raman spectrum of the label molecule gradually decreases. The characteristic Raman peak of the label molecule remains clearly distinguishable relative to the background signal even when the antigen concentration is lowered to 10-6 mg/mL, which represents the detection limit for the target antigen in this scheme.

FIG. 12 presents an upconversion spectrum obtained by performing luminescence detection on the substrate after an immunoassay reaction. The reaction involved the NaYF₄:Yb, Er upconversion luminescent nanoparticle immunoprobe and the MXene/MoS₂ composite SERS immunoassay substrate prepared in the present example, reacting with various concentrations of the target antigen (ranging from 10-2 mg/mL to 10-6 mg/mL). As can be seen from the figure, as the concentration of the target antigen decreases, the upconversion emission spectrum intensity gradually decreases. When the target antigen concentration is reduced to 10-6 mg/mL, the upconversion emission peak remains clearly distinguishable relative to the background signal, which represents the detection limit for the target antigen in the present scheme.

As can be understood from the foregoing examples and accompanying drawings, the preparation method for the dual-mode immunoassay structure based on a titanium carbide SERS substrate and rare-earth doped sodium yttrium fluoride nanoparticles, as provided by the present invention, demonstrates high detection sensitivity and high detection efficiency in application. The invention can be used for cancer screening, is critically important for understanding the course of cancer progression, and is suitable for widespread clinical application.

Furthermore, depending on the specific antigen, the dual-mode immunoassay structure based on a titanium carbide SERS substrate and rare-earth doped sodium yttrium fluoride nanoparticles provided by the present invention is not only suitable for the detection of prostate cancer but can also be applied to the detection of other cancers, such as colorectal cancer, ovarian cancer, or pancreatic cancer.

Those skilled in the art should understand that the preceding description and the embodiments shown in the accompanying drawings are for illustrative purposes only and are not intended to limit the present invention. The objectives of the present invention have been completely and effectively achieved. The function and structural principles of the present invention have been shown and explained in the embodiments, and equivalents and modifications of the implementation of the present invention may be made without departing from said principles.

What is claimed is:

1. A method for preparing a dual-mode immunoassay structure based on a titanium carbide SERS substrate and rare-earth doped sodium yttrium fluoride nanoparticles, the method comprises the following steps:
(S10) preparing a titanium carbide powder sing an acid-etching method;
(S20) preparing a titanium carbide/molybdenum disulfide composite immunoassay substrate; and
(S30) preparing a NaYF4:Yb,Er immunoprobe.

2. The method, as recited in claim 1, wherein the step (S10) comprises the following steps:
(S101) preparing a hydrochloric acid solution containing lithium fluoride;
(S102) adding titanium aluminum carbide powder to the solution of (S101) and reacting to form a titanium carbide solution;
(S103) centrifugally washing the titanium carbide solution with dilute hydrochloric acid and then with deionized water, followed by ultrasonication of the washed solution in an ice bath and centrifugation to obtain a supernatant; and
(S104) drying the supernatant to obtain the titanium carbide (MXene) powder.

3. The method, as recited in claim 2, wherein in step (S101), 1-3 g of lithium fluoride is added to 20 mL of 9 M hydrochloric acid and stirred for 10 minutes at room temperature; in step (S102), 1-3 g of titanium aluminum carbide powder is slowly added with stirring into the solution containing lithium fluoride and hydrochloric acid, and the mixture is heated in a water bath to 45° C. to react for 24-48 hours, thereby obtaining the titanium carbide solution; and in step (S103), the titanium carbide solution is centrifugally washed three times with 1 M dilute hydrochloric acid, followed by repeated washing with deionized water until a pH of the supernatant is between 6 and 7.

4. The method, as recited in claim 1, wherein the step (S20) comprises the following steps:
(S201) dissolving ammonium molybdate and thiourea in deionized water, adding titanium carbide, and ultrasonically dispersing to form a mixed solution;
(S202) placing the mixed solution in a Teflon-lined stainless-steel autoclave, heating to react, and upon completion, cooling and centrifuging to obtain a black precipitate;
(S203) washing the black precipitate with ethanol and deionized water and vacuum drying to obtain a titanium carbide/molybdenum disulfide (MXene/MoS₂) composite;
(S204) adding the MXene/MoS₂ composite to deionized water, depositing the resulting solution onto a silicon wafer, soaking the silicon wafer in DMF, and washing with a phosphate buffer solution;
(S205) adding a phosphate buffer solution containing NHS and EDC onto the titanium carbide/molybdenum disulfide composite formed on the silicon wafer; and
(S206) adding a solution containing an antibody, incubating, and washing to remove excess unreacted antibody to obtain the MXene/MoS₂ composite immunoassay substrate.

5. The method, as recited in claim 2, the step (S20) comprises the following steps:
(S201) dissolving ammonium molybdate and thiourea in deionized water, adding titanium carbide, and ultrasonically dispersing to form a mixed solution;
(S202) placing the mixed solution in a Teflon-lined stainless-steel autoclave, heating to react, and upon completion, cooling and centrifuging to obtain a black precipitate;
(S203) washing the black precipitate with ethanol and deionized water and vacuum drying to obtain a titanium carbide/molybdenum disulfide (MXene/MoS₂) composite;
(S204) adding the MXene/MoS₂ composite to deionized water, depositing the resulting solution onto a silicon wafer, soaking the silicon wafer in DMF, and washing with a phosphate buffer solution;
(S205) adding a phosphate buffer solution containing NHS and EDC onto the titanium carbide/molybdenum disulfide composite formed on the silicon wafer; and
(S206) adding a solution containing an antibody, incubating, and washing to remove excess unreacted antibody to obtain the MXene/MoS₂ composite immunoassay substrate.

6. The method, as recited in claim 4, wherein in step (S201), the ammonium molybdate is 15-20 mg, the thiourea is 30-50 mg, and the titanium carbide is 15-25 mg; in step (S202), the mixed solution is heated to 200° C. for 24 hours in the Teflon-lined stainless-steel autoclave and subsequently cooled to room temperature; and in step (S203), the black precipitate is washed 4-6 times with ethanol and deionized water and vacuum dried at 60° C. for 12 hours.

7. The method, as recited in claim 6, wherein in step (S204), the MXene/MoS₂ composite is added to deionized water at a mass-to-volume ratio of 1 mg: 100 μL to form the solution, 10-30 μL of which is deposited on the silicon wafer, and the silicon wafer is soaked in DMF for 2 hours; in step (S205), 1 mL of the phosphate buffer solution containing NHS and EDC at a 1:1 ratio and a concentration of 10 mg/mL is added; and in step (S206), 10 μL of a solution containing a PSA antibody is added, incubated at room temperature for 2 hours, and stored at 4° C. after washing.

8. The method, as recited in claim 1, wherein the step (S30) comprises the following steps:
(S301) mixing and stirring a rare-earth nitrate RE(NO₃)₃·6H₂O with an aqueous solution of citric acid;
(S302) adding a sodium hydroxide solution, stirring, and then adding a sodium fluoride solution to form a colloidal suspension;
(S303) transferring the colloidal suspension to a Teflon-lined stainless-steel reactor for reaction, and upon completion, cooling and centrifuging to obtain a reactant product;
(S304) washing the product with ethanol and deionized water, and drying;
(S305) mixing a solution of NaYF₄:Yb,Er in an R6G solution to react;
(S306) washing to remove excess R6G, adding a phosphate buffer solution of NHS and EDC, and incubating; and
(S307) rinsing with phosphate buffer, adding a solution containing an antibody, incubating, and washing to remove excess unreacted antibody, thereby obtaining the NaYF₄:Yb,Er (78% Y, 20% Yb, 2% Er) immunoprobe.

9. The method, as recited in claim 8, wherein: in step (S301), the rare-earth nitrate is RE(NO₃)₃·6H₂O, wherein RE comprises 78% Y, 20% Yb, and 2% Er; in step (S302), after being stirred for an additional 30 minutes, the colloidal suspension is transferred to the reactor and reacted at 180° C. for 12 hours; in step (S304), the product is washed 6 times and dried at 80° C. for 12 hours; in step (S305), the NaYF₄:Yb,Er solution is reacted in 5-10 mL of a 1 mM R6G solution for 15 minutes; in step (S306), the ratio of NHS to EDC is 1:1 at a concentration of 10 mg/mL; and in step (S307), a solution containing a PSA antibody is added and incubated for 1 hour.

10. The method, as recited in claim 8, further comprising a step of (S40): assembling a cancer biomarker detection system which comprises the following steps:
contacting the MXene/MoS₂ composite immunoassay substrate with a phosphate buffer solution containing an antigen to be measured to allow for an immunoassay reaction between the antigen and the antibody on the substrate, and washing to remove excess unreacted antigen; and
contacting the substrate with the NaYF₄:Yb,Er immunoprobe, reacting at 37° C., and washing to remove excess unreacted NaYF₄:Yb,Er immunoprobe, thereby forming the a cancer biomarker detection system.
